(19) United States
(12) Patent Application Publication
(10) Pub. No.: US 2010/0179413 A1
(43) Pub. Date: Jul. 15, 2010

(54) DETERMINATION AND DISPLAY OF MATERIAL PROPERTIES

(75) Inventors: Michael Joseph Kadour, Oxford (GB); Julia Alison Noble, Oxford (GB)

Correspondence Address: NIXON & VANDERHYE, PC, 901 NORTH GLEBE ROAD, 11TH FLOOR, ARLINGTON, VA, 22203, US

(73) Assignee: ISIS INNOVATION LIMITED, Oxford (GB)

(21) Appl. No.: 12/452,290

(22) PCT Filed: Jun. 25, 2008
(86) PCT No.: PCT/GB2008/002172

§ 371 Date: Feb. 23, 2010

(30) Foreign Application Priority Data
Jun. 26, 2007 (GB) 0712432.4

(51) Int. Cl. A61B 5/055; A61B 8/00

(52) U.S. Cl. 600/411; 600/438; 600/443

(57) ABSTRACT

The invention provides for the visualisation of conventional and parametric images of materials as they are progressively distorted during examination. A conventional image is displayed simultaneously alongside one or more parametric images derived from the original image data, with the parametric images displaying mechanical properties such as elasticity and mobility. The mobility values are calculated from the tracking error obtained from a motion or strain estimation algorithm applied to a sequence of image frames. The values of elasticity and mobility are displayed in a colour overlay on the conventional image background and the transparency of the overlay is varied according to the parameter values to de-emphasise less relevant values.

The present invention relates to the determination of mechanical properties of material, particularly of materials such as human or animal tissue, and the biomechanical properties of such tissue useful in clinical diagnosis. It also relates to improvements in the display of position-dependent properties of materials, especially in the visualisation of such properties.

A large variety of techniques are known for measuring various mechanical properties of materials, for example in the field of non-destructive testing and also in the clinical field as an aid to clinical diagnosis. In many such techniques the properties of the material which are measured can be displayed as an image (which may be two, three or four dimensional) and such images may be used as a basis for diagnosis along with other quantitatively or qualitatively measured properties. Over the years there have been continuous developments in the measurement of new material properties, and also in techniques for assisting the operator to interpret such information, for example by improvements in “visualisation”, meaning the visual display of those properties.

One such technique which is useful in many fields (including non-destructive testing and human and veterinary clinical practice) is ultrasound imaging. Ultrasound imaging is very well known and ultrasound data is typically displayed as a grey scale image representing a section in the depth direction (axial direction) through the material under examination. In the course of an ultrasound examination an ultrasound probe is brought into contact with the tissue under examination. In the clinical field it is common practice for ultrasonographers to use the probe to distort the tissue by pushing it into the tissue and observe how the tissue structures visible in the ultrasound image move and change. Thus in addition to a static image of the tissue, the reaction of the tissue to motion can be observed. This can assist in determining the nature of structures observed in the image.

It has also been proposed to calculate from a sequence of images of the tissue undergoing deformation the elasticity (or more accurately the axial strain of the tissue). It should be noted that in this specification the term elasticity will be used for the change in length per original length, as it is the term of the art in medical imaging, though to be technically accurate, for example in materials science, this would be referred to as strain. Techniques for calculating elasticity from ultrasound data are disclosed, for example, in the following documents incorporated herein by reference:
[1] Ophir J, Cespedes I, Ponnekanti H, Yazdi Y, and Li X, “Elastography: a quantitative method for imaging the elasticity of biological tissues,” Ultrason. Imaging, vol. 13, pp. 111-34, 1991.
[2] Gao L, Parker K J, Lerner R M, and Levinson S F, “Imaging of the elastic properties of tissue—A review,” Ultrasound Med. Biol., vol. 22, pp. 959-77, 1996.
[3] Garra B S, Cespedes E I, Ophir J, Spratt S R, Zuurbier R A, Magnant C M, and Pennanen M F, “Elastography of breast lesions: Initial clinical results,” Radiology, vol. 202, pp. 79-86, 1997.
[4] Varghese T, Ophir J, and Cespedes I, “Noise reduction in elastograms using temporal stretching and multicompression averaging,” Ultrasound Med. Biol., vol. 22, pp. 1043-52, 1996.
[5] Hein I A and Obrien W D, “Current time-domain methods for assessing tissue motion by analysis from reflected ultrasound echoes—A review,” IEEE Trans. Ultrason. Ferroelectr. Freq. Control, vol. 40, pp. 84-102, 1993.
[6] Kallel F and Ophir J, “A least-squares strain estimator for elastography,” Ultrason. Imaging, vol. 19, pp. 195-208, 1997.
[7] Viola F and Walker W F, “A spline-based algorithm for continuous time-delay estimation using sampled data,” IEEE Trans. Ultrason. Ferroelectr. Freq. Control, vol. 52, pp. 80-93, 2005.
[8] U.S. Pat. No. 5,107,837

Such elasticity information can conveniently be displayed to the operator as a colour overlay on the conventional grey scale ultrasound image with different colours corresponding to different values of elasticity.

However, elasticity does not always resolve ambiguities seen in the basic ultrasound image, and it has also been found that the use of the colour overlay on the conventional grey scale ultrasound image can be distracting for the operator and cause them to miss significant features.

A first aspect of the present invention relates to the quantification of a further mechanical property of the material in an object undergoing examination which is of great use in providing additional information and removing ambiguity between images of different structures which have similar stiffness (e.g. in the clinical field for example fibroadenomas and tumours). In more detail it relates to a method of quantifying the mobility of structures, namely the degree to which one region of material moves independently from another. This is achieved by inducing a movement in the material and differentiating regions in the image which move in a uniform and connected manner from those which move irregularly or independently from neighbouring regions. The quantified measure of mobility is here called “slip”, there being a “slip value” for each of a plurality of parts of the material under examination.

As a first aspect therefore, the invention provides a method of obtaining quantified slip values of material in an object undergoing an imaging examination to provide a sequence of images of the material, comprising physically distorting the material, measuring the resultant motion of the material through the sequence of images to estimate the degree of relative mobility of different areas of the material, and calculating the slip values on the basis of said resultant motion.

The examination may be an ultrasound examination, e.g. B-mode, or may be an MRI or other imaging modality, to provide a primary image from which the slip values may be calculated.

The slip values may be displayed visually superimposed on the primary image of the material, for example with different slip values corresponding to different values of an attribute of an image overlay visually superimposed on the primary image. The image attribute which varies can be the colour or intensity.

In addition, the transparency of the image overlay may be varied depending on the slip value so that the overlay is more transparent where slip values are closer to those expected of normal material. This means that not only does the colour change according to the slip value, but also that the colour overlay is stronger where the slip values are abnormal and more transparent where they are normal, which helps the operator direct attention to regions of interest. Preferably the transparency varies progressively depending on the slip value. The dependency may be user-selectable so that low transparency can be set to correspond to whatever slip values are of most interest. In the field of breast ultrasound examination for breast cancer, for example, benign objects of interest such as cysts and some hard solid lesions tend to have high slip values (high mobility) and so the image overlay is made less transparent for areas of high slip value, thus providing a contrast with hard malignant structures which tend to have low slip values (low mobility).

The slip values are based on the degree of relative mobility which preferably is the non-uniformity of movement or the amount of movement of a structure relative to neighbouring regions. This may be calculated from a motion field which in turn is calculated from a sequence of images taken as the material is deformed. Such a motion field can be calculated using a conventional motion estimation algorithm. Preferably the estimate of the degree of mobility and the slip values are calculated from the quality of the tracking achieved by the motion estimation algorithm. Thus regions where the quality of the tracking is high correspond to low mobility material, whereas regions where the quality of the tracking is low correspond to high mobility material. Preferably the quality of the tracking is taken as the tracking error returned by the motion estimation algorithm. This of course assumes a minimum level of tracking quality is obtained for a sufficient portion of the region of interest. If the quality of the tracking overall is insufficient, it may be more appropriate to indicate that general tracking failure or excessive noise occurred since in this situation the correspondence between tracking quality and tissue mobility will be minimized.

The estimate of the degree of mobility may be calculated as a linear sum or any other function of the estimated tracking errors returned by the motion estimation algorithm, or of the image correlation of the regions tracked by the motion estimation algorithm.

The estimate of the degree of mobility may be calculated from a strain field calculated from a sequence of images taken as the material is deformed, e.g. using an elasticity strain estimation algorithm, and more particularly from the quality of the strain estimation achieved by the strain estimation algorithm or from the strain estimation error returned by the strain estimation algorithm.

The estimate of the degree of mobility may be calculated from a combination of at least two of the estimation qualities achieved by the motion estimation algorithm, the strain estimation algorithm, and image correlation.

The deformation of the material may be applied manually by the operator, or by means of a machine which provides automated incremental displacements of the material to provide contact force externally at the surface. In the case of an ultrasound examination, the force may be applied by using the ultrasound probe itself to distort the material or by utilising acoustic force in the material due to the ultrasound propagation. The deformation of the material may also be achieved by natural sources of motion, such as, in a biological subject: breathing or cardiac pulse.

A second aspect of the invention relates to improving the visualisation of position-dependent properties of a subject. Accordingly, this aspect of the invention provides a method of displaying measurements of a plurality of different position-dependent properties of a subject, comprising displaying a primary image representing a first of said position-dependent properties, and displaying visually superimposed over the primary image an overlay image whose image attributes vary to represent the values of a second of said position-dependent properties, and wherein the transparency of the overlay image spatially varies progressively in dependence on the value of said second of said position-dependent properties.

Thus two position-dependent properties can be displayed, one in the primary image and one as an overlay image which is visually superimposed. To avoid confusion in the image, the overlay image is rendered transparent in certain areas, preferably where the values of the second of the position-dependent properties are of low interest (for example where the values are normal). Thus, preferably, areas of the overlay image representing normal values of the second of the position-dependent properties are given higher transparency than areas representing interesting or abnormal, e.g. clinically relevant, values.

The overlay image may contain areas which are completely transparent, i.e. effectively the values of the second of the position-dependent properties are not shown. But in areas which are not completely transparent, the transparency varies according to the values of the second of the position-dependent properties.

The relationship between the transparency and the values of the second of the position-dependent properties may be user-selectable so that the user can adapt the display to different applications. The attribute of the overlay image which varies to represent the values of the second of the position-dependent properties can be the colour or intensity, though colour is particularly effective when overlaid on a grey scale image such as an ultrasound image.

The second of the position-dependent properties may be a mechanical property, for example elasticity or mobility. The second of the position-dependent properties can be the elasticity of the material, with areas of high stiffness corresponding to low transparency. Alternatively, the second of the position-dependent properties can be slip values representing estimates of the mobility of the material, with areas of high mobility corresponding to low transparency.

It is particularly effective if the original image and composite images consisting of original image with the overlay are displayed alongside each other, preferably with the same scale and area, allowing easy comparison.

Thus a third aspect of the invention provides a method comprising displaying adjacent to each other a first image consisting of a primary image of material under examination, a second image representing the elasticity of the material under examination, and a third image displaying slip values representing estimates of the mobility of different areas of the material under examination.

The primary image can be an ultrasound image, or MRI or other medical imaging modality.

As indicated above, preferably the three images are displayed with the same scale and area, and the second and third images may be two composite images including overlays indicating the values of additional properties of the material such as elasticity (strain) and slip (mobility). Preferably the transparency of the overlays in the composite images is varied as mentioned above.

The images displayed in the three aspects above can be still or video images. As video images, they may be displayed in real-time, or pre-recorded and played back. Further, although the invention is exemplified above and below in the field of ultrasound, the invention is applicable to other imaging modalities.

The images of the object may be in 1, 2 or 3 dimensions and the subject may be any biological, e.g. human, animal or plant, or non-biological, material.

The invention is conveniently embodied in software for processing ultrasound image data and thus the invention extends to a computer program comprising program code means for executing the methods above on a programmed computer. The invention also extends to an ultrasound imaging apparatus including a processor adapted to process ultrasound signals and to display them as set out above.

The invention will be further described by way of non-limitative example with reference to the accompanying drawings in which:

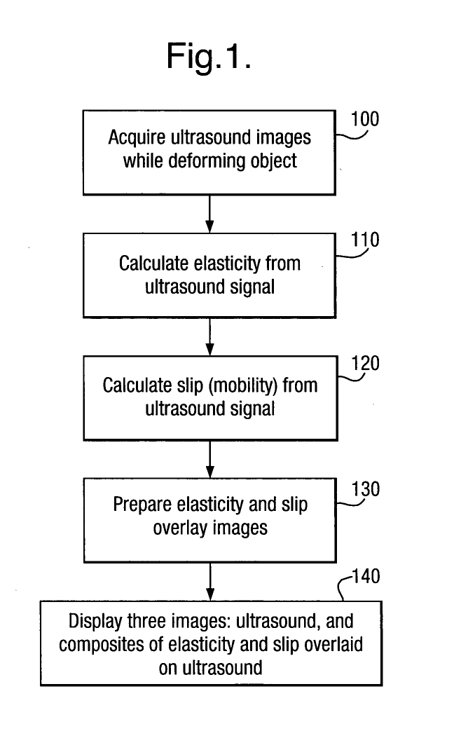

FIG. 1 is a flow diagram showing the overall steps in one embodiment of the invention;

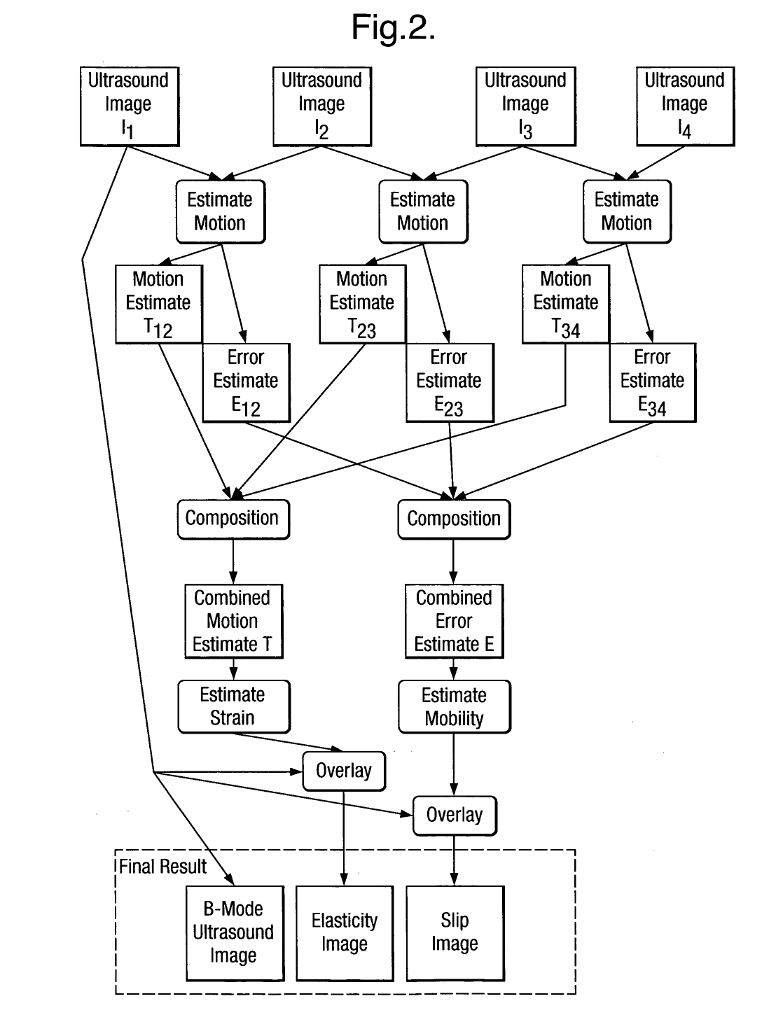

FIG. 2 is a flow diagram showing the steps of the embodiment of FIG. 1 in more detail;

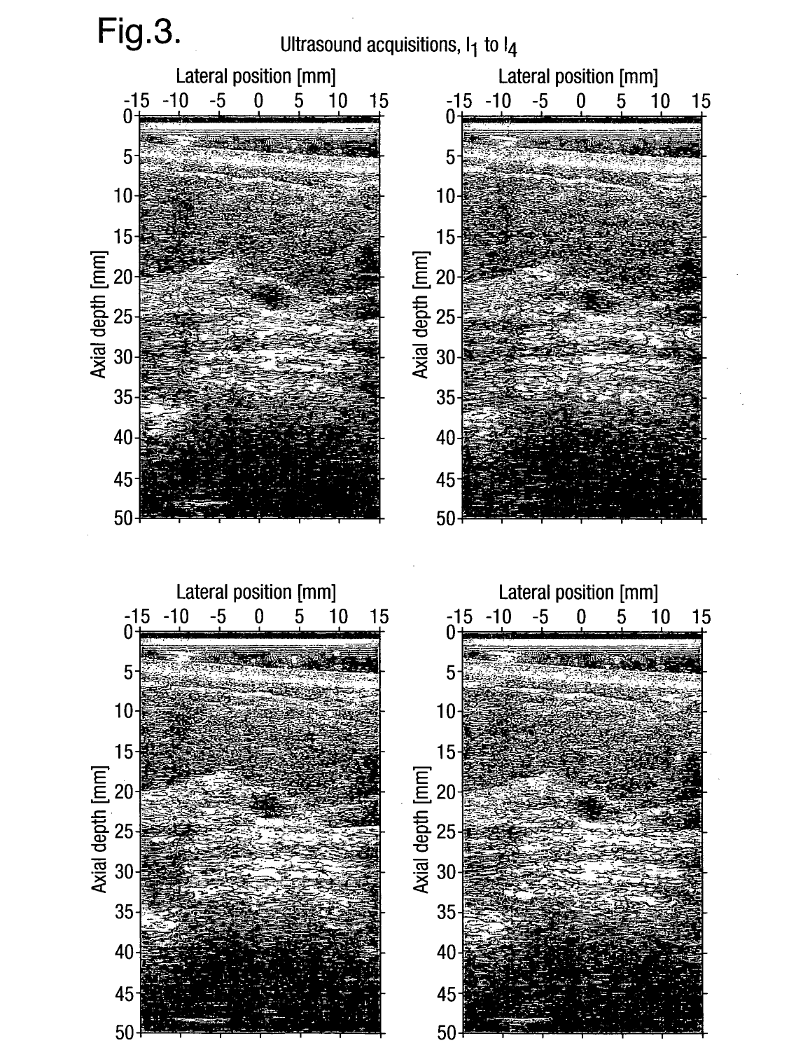

FIG. 3 illustrates four sequential images from an ultrasound acquisition used in an example of the invention;

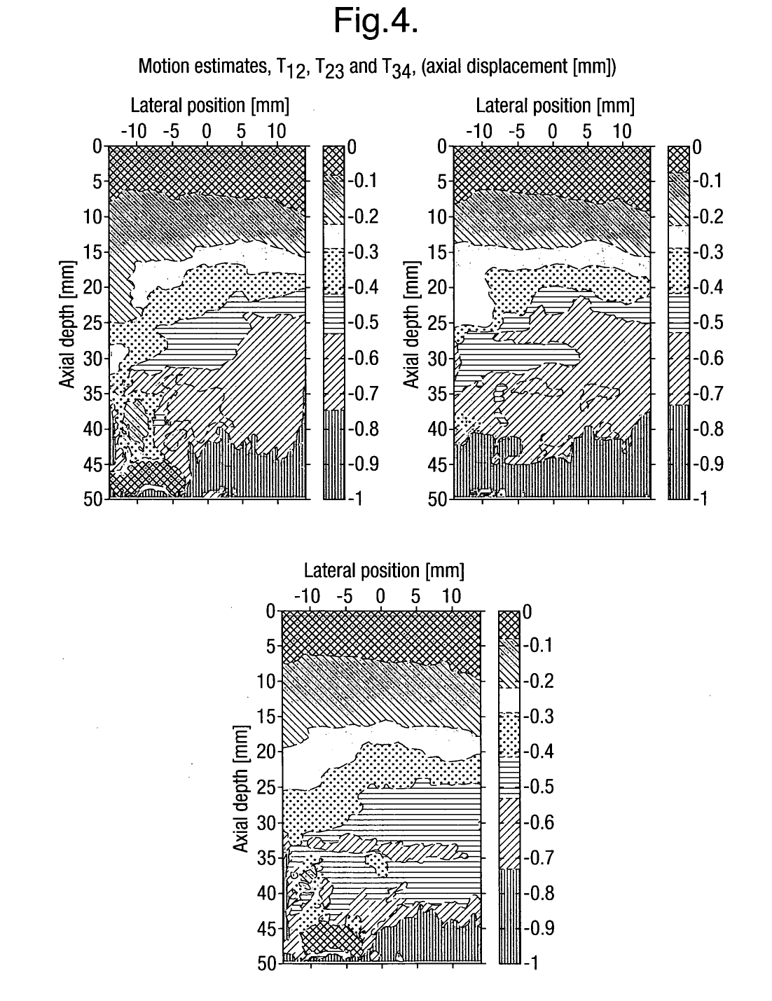

FIG. 4 illustrates three motion fields calculated from the ultrasound images of FIG. 3;

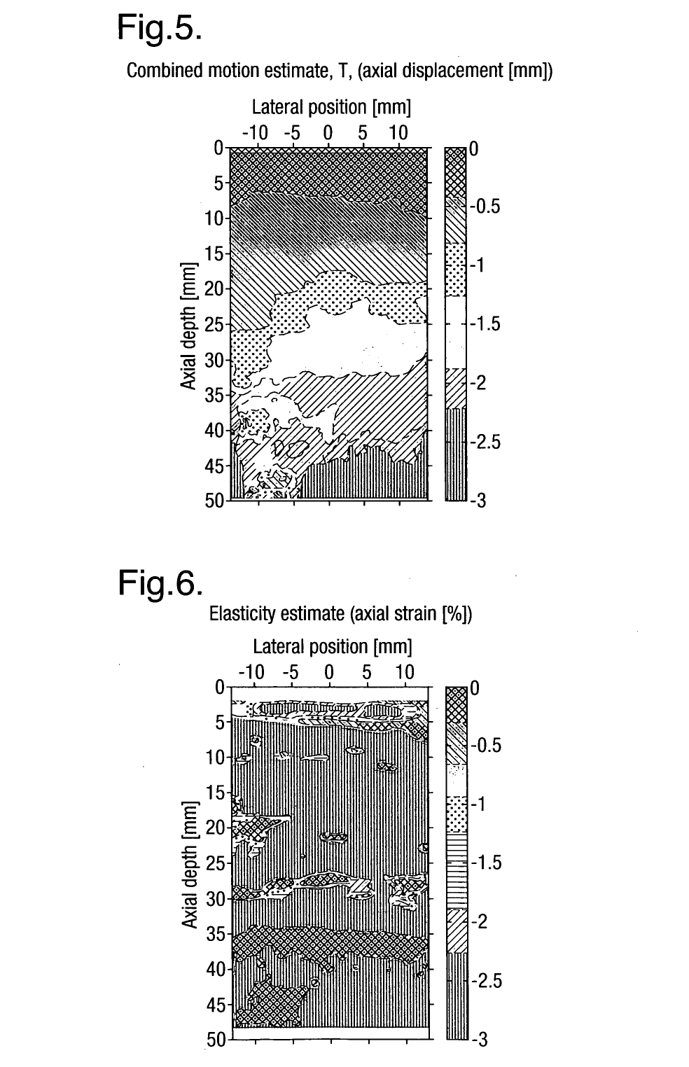

FIG. 5 illustrates a combined motion field obtained from the three motion fields of FIG. 4;

FIG. 6 illustrates an elasticity estimate obtained from the combined motion field of FIG. 5;

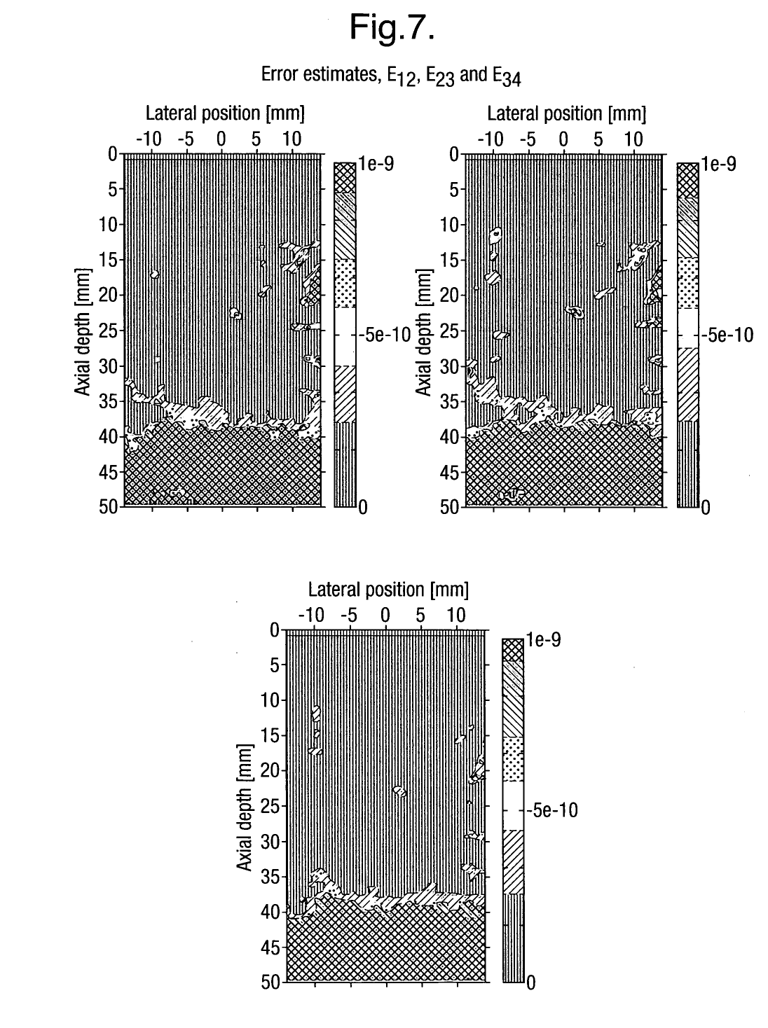

FIG. 7 illustrates the three error fields in the motion estimates of FIG. 4;

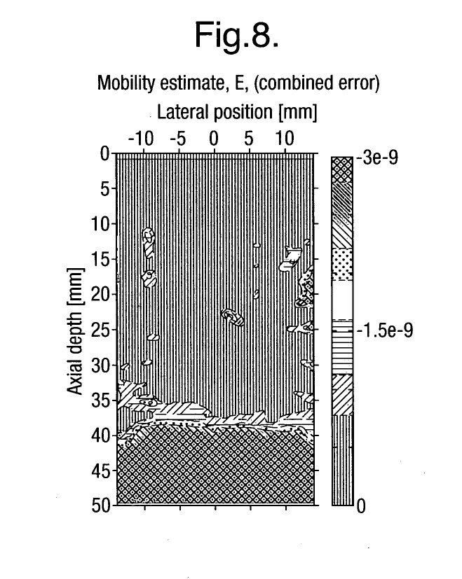

FIG. 8 illustrates the combined mobility estimate obtained from the three error fields of FIG. 7;

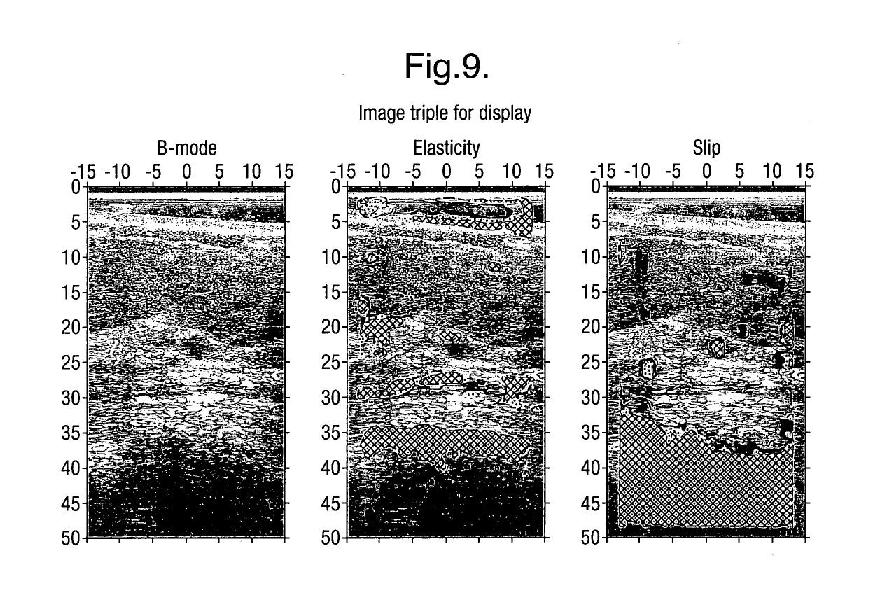

FIG. 9 illustrates three images, the original and two composite images being displayed alongside each other; and

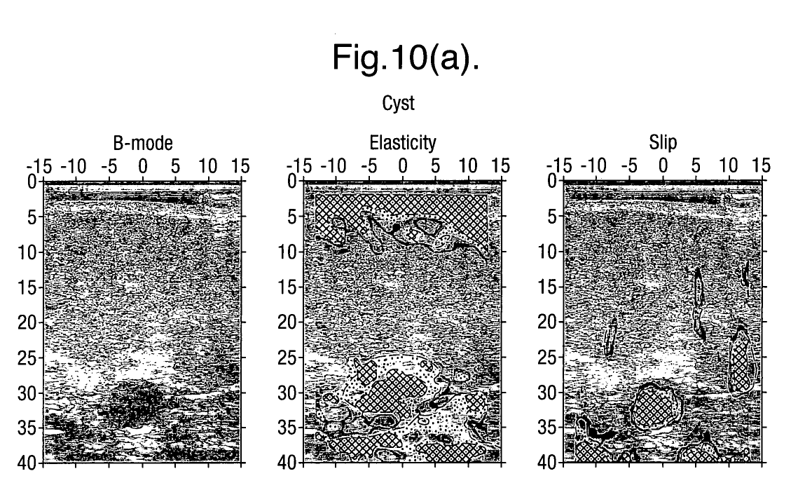

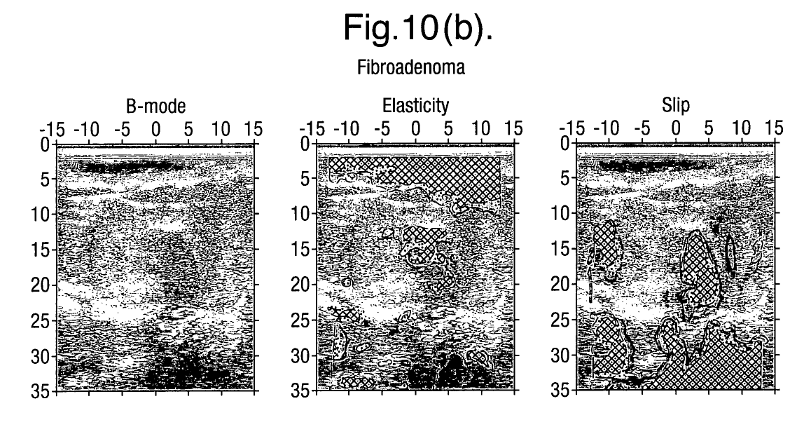

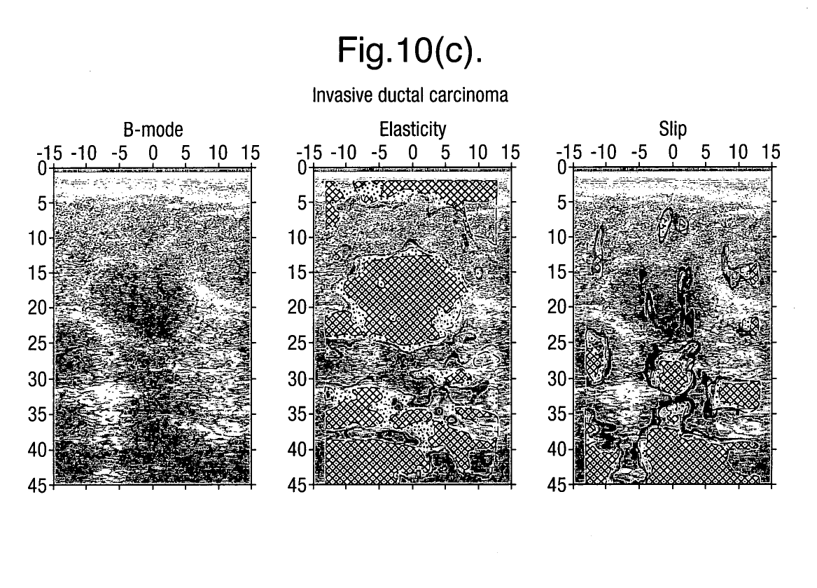

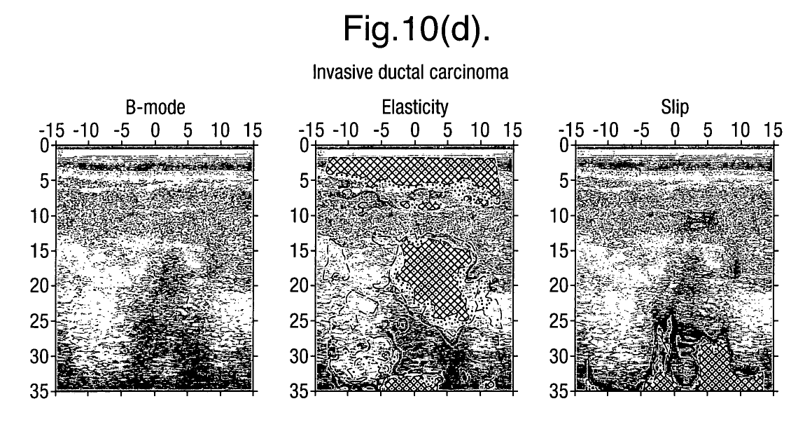

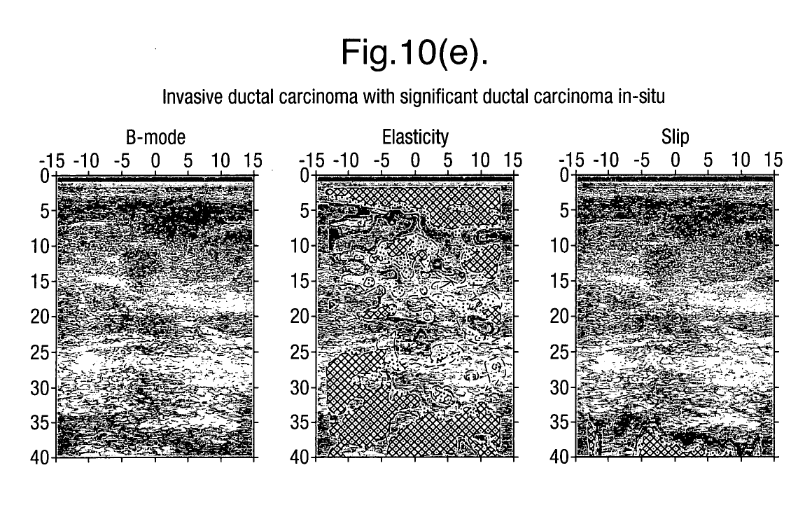

FIGS. 10(a) to (e) show similar image triples for tissue including different structures of interest.

FIG. 1 illustrates the overall process of one embodiment of the invention as applied to ultrasound imaging of a human being. In step 100, ultrasound data is recorded using an ultrasound scanning machine in the conventional manner by a radiologist holding an ultrasound probe against the part of the anatomy to be imaged. The ultrasound data is obtained while progressively deforming the tissue by compressing the tissue in the axial direction (perpendicular to the face of the ultrasound probe) with the probe (or alternatively by gradually decompressing the tissue). The elasticity of the tissue is calculated in step 110 by a conventional technique such as one of those disclosed in the references listed above. Also, in step 120 the slip values (quantitative measurements of mobility) are also calculated from the ultrasound signal. One way of doing this is described below.

The elasticity and slip will be visualised by displaying them as coloured image overlays on the original ultrasound image. Thus, in step 130 the overlays are prepared from the elasticity and slip data and in particular the transparencies of the overlays are varied with the clinically relevant regions (high stiffness and high slip) made more opaque (less transparent) and the less relevant regions (low stiffness, low slip) made more transparent. Finally, in step 140 an image triple consisting of the original ultrasound image with two composite images alongside it are displayed, the two composite images being the basic ultrasound image with the elasticity information overlaid and the basic ultrasound image with the slip values overlaid. The image triple gives a more complete picture of the tissue properties and can increase the confidence in, and power of, ultrasound imaging diagnosis as will be illustrated below by some specific examples.

A specific example will now be described with reference to an ultrasound acquisition of four sequential images.

The invention is applicable to conventional methods of ultrasound elasticity imaging as set out, for example, in the publications by J Ophir et al., L Gao et al. or B S Gara et al. mentioned above. Deriving the mobility information (slip values) requires at least two ultrasound images, but preferably more to reduce estimation noise as described in the paper by T Varghese et al. referenced above. Thus in general, an ultrasound acquisition of N sequential images, I₁-I_{N}, is made while the tissue is slowly compressed in the axial direction of the ultrasound scan plane (though alternatively the deformation can be a decompression). Motion is then estimated between each consecutive pair of ultrasound images using a conventional motion algorithm such as sum-of-squared differences block-matching as described in the paper by I A Hein and W D O'Brien referenced above. Non-consecutive image pairs can be also used, with the pairs selected to improve the overall quality of the motion estimation. Other motion estimation algorithms are, of course, available and can be used preferably if they have an associated error or quality output.

In this particular algorithm a sum-of-squared-differences error:

E₁₂(x)=(I₂(T₁₂(x)+x)−I₁(x))²

is minimized over multiple discrete image regions (blocks) to find the optimal motion estimate:

T₁₂(x)=argmin(E₁₂(x)),

where x is the spatial coordinate and can be in multiple dimensions.

This results in N−1 motion field estimates, denoted T₁₂ through T_{(N-1) N}, and N−1 error estimates, denoted E₁₂ through E_{(N-1)N}, between the successive image pairs.

If N>2, the multiple motion and error estimates are combined into single estimates:

T=T₁₂+T₂₃(T₁₂+x)+T₃₄(T₂₃(T₁₂+x)+T₁₂+x)+ . . . ,

E=E₁₂+E₂₃(T₁₂+x)+E₃₄(T₂₃(T₁₂+x)+T₁₂+x)+ . . . .

The compositions can be obtained by interpolating each motion and error field at the locations specified by the previous motion fields. Additionally, the error fields can be combined using any function instead of the linear summation shown.

The composed motion field is used to derive an elasticity estimate according to a conventional axial strain estimation algorithm such as a least-squares gradient estimator.

The composed error field is used as the estimate of mobility (slip values). Alternatively, if estimate quality, Q, is available instead of an estimate error, it can be used by subtracting it from its maximum value:

Eᵢⱼ=max(Q)−Qᵢⱼ

In this manner, the quality or similarity metric can be converted and then composed like the error metric to be used as an estimate of mobility.

The results are presented in an image triple with the ultrasound B-mode, elasticity, and slip images (step 140). Ultrasound B-mode is a grey scale image and is used as the background for all three images. The elasticity and slip images are created by overlaying the estimated elasticity and mobility data as colour over the ultrasound B-mode image background. In the attached drawings to be published, to assist viewing in monochrome, the different coloured regions have been arbitrarily outlined and shaded so that they can be seen, but in the originally filed drawings and in practice there are no such outlines—just a progressively varying colour and varying transparency overlay.

Depending on the application, different elasticity and mobility values have increased importance. For instance, in breast tissue diagnosis hard tissue often correlates to malignancy, and highly mobile tissue often correlates to benignity. To highlight the features of interest, the harder and more mobile regions are made redder and more opaque, whereas the softer and less mobile regions are made bluer and more transparent. Thus this spatially varying transparency is a function of the elasticity or slip value according to some arbitrary mapping function which can be a simple linear mapping.

### EXAMPLE

The data flow for this example is shown in FIG. 2.

### Step 1

Four ultrasound images, shown in FIG. 3, were recorded while the tissue was being compressed a total of 3 mm. The Analogic AN2300 ultrasound engine was used with the BK 8805 5-12 MHz small parts ultrasound transducer to record this data with each frame 30 mm wide (laterally) and 50 mm deep (axially). The imaged breast tissue contains a small 5 mm by 3 mm breast cyst located 1 mm to the right of center laterally and 22 mm deep. The compression between each frame was only 1 mm so the four images look very similar. Compressions between 0.1 mm and 1 mm provide sufficient contrast between those regions which are highly mobile and those which are less mobile.

### Step 2

With the four ultrasound images (FIG. 3) the motion was estimated between each consecutive pair using a sum-of-squared-differences block-matching algorithm (reference 5 above). The block size was 1.5 mm by 1.5 mm with an overlap of 87.5% in both axial and lateral directions. Sub-sample accuracy was obtained using spline-interpolation (reference 7 above). This resulted in three motion fields, T₁₂, T₂₃, and T₃₄, of which the axial (depth) components are shown in FIG. 4, and three error fields, E₁₂, E₂₃, and E₃₄, which are shown in FIG. 7.

### Step 3

The three motion fields (FIG. 4) and three error fields (FIG. 7) must be composed into single fields. The process of composition is the same for the motions and the errors. Taking the motion fields first, the goal is to have a single composed motion field in the reference frame of the first ultrasound image. The three motion fields, T₁₂, T₂₃, and T₃₄ were composed in the following way:

T=T₁₂+T₂₃(T₁₂+x)+T₃₄(T₂₃(T₁₂+x)+T₁₂+x).

This required T₂₃ to be interpolated at the locations specified by T₁₂, and T₃₄ to be interpolated at the locations specified by T₂₃ and T₁₂. Spline interpolation was used for this task. In a similar manner the composed error field, E, was calculated:

E=E₁₂+E₂₃(T₁₂+x)+E₃₄(T₂₃(T₁₂+x)+T₁₂+x).

This required E₂₃ and E₃₄ to be interpolated like T₂₃ and T₃₄, and spline interpolation was also used for this task. Thus the three motion fields shown in FIG. 4 produced the single motion field shown in FIG. 5, and the three error fields shown in FIG. 7 produced the single error field shown in FIG. 8.

### Step 4

The elasticity image is obtained by calculating the gradient of the motion image, or how much areas have changed in size (i.e. the strain). Thus using the composed motion field of FIG. 5, the axial strain was estimated using a least-squares gradient estimator (reference 6 above). The block size was 3 mm axially and 1 mm laterally with a maximal overlap (i.e. the strain was estimated at every motion estimate). The axial strain estimate is shown in FIG. 7.

### Step 5

Three images were created for the final result shown in FIG. 9. The first ultrasound image, I₁, (FIG. 3) was used for the first final image. The axial strain estimate (FIG. 7) was overlaid on the first ultrasound image to create the elasticity image; the second of the three final images. The transparency was set to 1 (fully transparent) for the regions with maximum strain (softest tissue) and 0.5 (partially transparent) for the regions with minimal strain (hardest tissue). The composed error field (FIG. 8) was overlaid on the first ultrasound image to create the slip (mobility) image; the last of the three final images. The transparency was set to 1 (fully transparent) for the regions with minimal mobility (fixed normal tissue) and 0.5 (partially transparent) for the regions with maximal mobility (fluid or freely moving tissue).

FIGS. 10a to e illustrate image triples obtained in accordance with the procedure above for tissues containing different structures of interest. FIG. 10a shows images of a cyst with a soft (low elasticity), mobile (high slip) interior. FIG. 10b shows images of a fibroadenoma with regions of stiffness (high elasticity), but also regions of high slip. FIGS. 10c, d and e show images of various malignant tumours, each of which has focal regions which are much stiffer (higher elasticity) than the background, but with no slip. It can be seen that the combination of the three different types of information assist in distinguishing the different tissue structures.

In the example above, the tracking error obtained from the motion estimation algorithm is used as a measure of slip (or mobility). In general any parameter which is indicative of areas where tracking is not good can be used. Typically low quality tracking is caused by out of plane motion or by movement of areas which are filled with fluid and thus are random or highly spatially-varying. Additionally, any parameter which is indicative of areas of increased mobility, that is areas with motion independent or greater than neighbouring areas, can also be used.

Although in the example above the transparency of the overlays is adjusted to lie between 0.5 (partially transparent) and 1 (fully transparent), the relationship can be different, or can be set differently for different applications. Control of this may be given to the user, who can then try different relationships between transparency and the values to choose one which gives the greatest distinctiveness of the regions of interest. The values above are chosen in order to de-emphasise normal responses from the tissue in order to avoid distracting the user from regions of interest.

1-50. (canceled)

51. A method of obtaining quantified slip values of material in an object undergoing an imaging examination to provide a sequence of images of the material, comprising physically distorting the material, measuring the resultant motion of the material through the sequence of images to estimate the degree of relative mobility of different areas of the material, and calculating the slip values on the basis of said resultant motion.

52. A method according to claim 51 further comprising displaying the slip values visually superimposed on the image of the material.

53. A method according to claim 52 wherein different slip values are displayed by different attributes of an image overlay visually superimposed on the image of the material.

54. A method according to claim 53 further comprising spatially varying the transparency of the image overlay depending on the slip value.

55. A method according to claim 54 wherein the transparency of the image overlay varies progressively depending on the slip value.

56. A method according to claim 55 wherein high slip values corresponding to high relative mobility of different areas of the material are represented with lower transparency.

57. A method according to claim 52 wherein the slip values are displayed on a sequence of frames forming a video sequence.

58. A method according to claim 51 wherein the degree of relative mobility of a region is the non-uniformity of movement of a region relative to neighbouring regions.

59. A method according to claim 51 wherein the degree of relative mobility of a region is the amount of movement of a region relative to neighbouring regions.

60. A method according to claim 51 wherein the material is tissue and the physical distortion of the tissue is due to the breathing of the subject.

61. A method according to claim 51 wherein the material is tissue and the physical distortion of the tissue is due to the cardiac pulse-induced motion of the subject.

62. A method according to claim 51 wherein the physical distortion of the material is due to the natural movements of the object.

63. A method according to claim 51 wherein the physical distortion of the material is due to an externally applied force.

64. A method according to claim 63 wherein the force is applied by an ultrasound probe performing an ultrasound examination through either contact force exerted at the surface or through acoustic force due to the ultrasound propagation.

65. A method according to claim 64 wherein the ultrasound probe is used freehand.

66. A method according to claim 64 wherein the material is progressively distorted by automated incremental displacements of the ultrasound probe.

67. A method according to claim 51 wherein the estimate of degree of mobility is calculated from a motion field calculated from a sequence of images taken as the material is deformed.

68. A method according to claim 67 wherein the motion field is calculated using a motion estimation algorithm.

69. A method according to claim 68 wherein the estimate of the degree of mobility is calculated from the quality of the tracking achieved by the motion estimation algorithm.

70. A method according to claim 68 wherein the estimate of the degree of mobility is calculated as a linear sum or any other function of the estimated tracking errors returned by the motion estimation algorithm.

71. A method according to claim 68 wherein the estimate of the degree of mobility is calculated from the image correlation of the regions tracked by the motion estimation algorithm.

72. A method according to claim 51 wherein the estimate of the degree of mobility is calculated from a strain field calculated from a sequence of images taken as the tissue is deformed.

73. A method according to claim 72 wherein the strain field is calculated using an elasticity strain estimation algorithm.

74. A method according to claim 73 wherein the estimate of the degree of mobility is calculated from the quality of the strain estimation achieved by the strain estimation algorithm.

75. A method according to claim 73 wherein the estimate of the degree of mobility is calculated from the strain estimation error returned by the strain estimation algorithm.

76. A method according to claim 51 wherein the estimate of the degree of mobility is calculated from a combination of at least two of the following: an estimate based on a strain field calculated from a sequence of images taken as the tissue is deformed; an estimate based on a motion estimation algorithm; and image correlation of regions tracked by the motion estimation algorithm.

77. A method according to claim 51 wherein the images of the object are in 1, 2 or 3 dimensions.

78. A method of displaying measurements of a plurality of different position-dependent properties of a subject, comprising displaying a primary image representing a first of said position-dependent properties, and displaying visually superimposed over the primary image an overlay image whose image attributes vary to represent the values of a second of said position-dependent properties, and wherein the transparency of the overlay image spatially varies progressively in dependence on the value of said second of said position-dependent properties.

79. A method according to claim 78 wherein the progressive dependence is such that areas of said overlay image representing normal values of said second of said position-dependent properties are given higher transparency than areas representing abnormal values of said second of said position-dependent properties.

80. A method according to claim 78 wherein the relationship between transparency and the values of said second of said position-dependent properties is user-selectable.

81. A method according to claim 78, wherein the image attribute of the overlay image which varies to represent the values of said second of said position-dependent properties is the colour.

82. A method according to claim 78, wherein the image attribute of the overlay image which varies to represent the values of said second of said position-dependent properties is the intensity.

83. A method according to claim 78 wherein the primary image is a gray scale image.

84. A method according to claim 78 wherein the primary image is an ultrasound, MRI or other medical image.

85. A method according to claim 78 wherein the second of said position-dependent properties is a biomechanical property of the tissue.

86. A method according to claim 78 wherein the second of said position-dependent properties is the elasticity of the tissue.

87. A method according to claim 86 wherein areas of low elasticity (low stiffness, high strain) correspond to high transparency of said image overlay.

88. A method according to claim 78 wherein the second of said position-dependent properties is slip value representing estimates of the degree of relative mobility of different areas of the tissue.

89. A method according to claim 88 wherein areas of low slip (low mobility) correspond to high transparency of said image overlay.

90. A method according to claim 78 wherein said primary image comprises a sequence of frames forming a video image.

91. A method according to claim 78 wherein said primary image and said secondary overlay image comprises a sequence of frames forming two temporally synchronized video images.

92. A method according to claim 78 wherein a first image consisting of the primary image alone, a second image consisting of the primary image with an image overlay representing the elasticity of the tissue, and a third image consisting of the primary image with an image overlay representing estimates of the degree of relative mobility of different areas of the tissue are displayed adjacent to each other.

93. A method comprising displaying adjacent to each other a first image of material under examination, a second image representing the elasticity of the material under examination, and a third image displaying slip values representing estimates of the degree of relative mobility of different areas of the material under examination.

94. A method according to claim 93 wherein the first, second and third images are displayed with the same scale and area.

95. A method according to claim 93 wherein the first, second and third images display parameters for a coincident physical region.

96. A method according to claim 93, wherein the second and third images are composites of the first image with an overlay image representing said elasticity and slip values visually superimposed thereon.

97. A method according to claim 93, wherein the first, second and third images comprises a sequence of frames forming three temporally synchronized video images.

98. A method according to claim 93, wherein only a subset of the first, second or third images is displayed.

99. A computer readable storage medium having tangibly encoded thereon program comprising program code means for executing on a programmed computer the method of claim 51.

100. An ultrasound imaging apparatus comprising an ultrasound image display and an ultrasound signal processor adapted to process an ultrasound signal in accordance with the method of claim 51.

101. A computer readable storage medium having tangibly encoded thereon program comprising program code means for executing on a programmed computer the method of claim 78.

102. An ultrasound imaging apparatus comprising an ultrasound image display and an ultrasound signal processor adapted to process an ultrasound signal in accordance with the method of claim 78.

103. A computer readable storage medium having tangibly encoded thereon program comprising program code means for executing on a programmed computer the method of claim 93.

104. An ultrasound imaging apparatus comprising an ultrasound image display and an ultrasound signal processor adapted to process an ultrasound signal in accordance with the method of claim 93.
